(19) United States
(12) Patent Application Publication
(10) Pub. No.: US 2005/0080299 A1
(43) Pub. Date: Apr. 14, 2005

(54) Method for the preparation of matairesinol

(75) Inventors: Rainer Sjoholm, Piispanristi (FI); Patrik Eklund, Turku (FI); Jyri Pekka Mikkola, Turku (FI); Reko Lehtila, Lohja (FI); Marja Sodervale, Oulu (FI); Arja Kalapudas, Oulu (FI)

Correspondence Address: JAMES C. LYDON, 100 DAINGERFIELD ROAD, SUITE 100, ALEXANDRIA, VA, 22314, US

(73) Assignee: Hormos Medical Corporation, FI-20520 Turku (FI)

(21) Appl. No.: 10/499,484

(22) PCT Filed: Jan. 2, 2003
(86) PCT No.: PCT/FI03/00001

(30) Foreign Application Priority Data
Jan. 9, 2002 (FI) 200220032
Feb. 6, 2002 (FI) 20020235

(51) Int. Cl. C07C 37/06

(52) U.S. Cl. 568/799

(57) ABSTRACT

A method for the preparation of matairesinol from hydroxymatairesinol, either by (i) catalytic hydrogenolysis of the hydroxy group in 7-position of hydroxymatairesinol, where the reaction is carried out in a suitable solvent as a pressurized hydrogenolysis, or (ii) reduction of hydroxymatairesinol, where the reduction is carried out as a hydrogen transfer reaction from a hydrogen donor in the presence of a catalyst.

### FIELD OF THE INVENTION

This invention relates to a novel method for the preparation of the plant lignan matairesinol.

### BACKGROUND OF THE INVENTION

The publications and other materials used herein to illuminate the background of the invention, and in particular, cases to provide additional details respecting the practice, are incorporated by reference.

Hydroxymatairesinol and matairesinol, the chemical structures of which are shown in Scheme 1, are both known biologically active plant lignans. Hydroxymatairesinol appears as two diastereomers, namely (−) hydroxymatairesinol (also denoted HMR 2 isomer) and (−) allo-hydroxymatairesinol (also denoted HMR 1 isomer).

EP 906761 discloses matairesinol as useful in a nutritional supplement for prevention cancers, coronary heart diseases and hormonal disorders. It is also known that matairesinol is a precursor for the mammalian lignan enterolactone (J D Ford et al., Plant Polyphenols 2: Chemistry, Biology, Pharmacology, Edited by Gross et al. Kluwer Academic/Plenum Publisher, New York 1999, pp. 675-694).

Matairesinol is, as many other lignans, found in different parts of plants and trees (roots, leafs, stem, seeds, fruits) but mainly in small amounts. In many sources (seeds, fruits) lignans are found as glycosidic conjugates associated with fiber component of plants. The most common dietary sources of mammalian lignan precursors are unrefined grain products. The highest concentrations have been found in flaxseed. The restricted availability of matairesinol in large quantities is a considerable problem which must be overcome before matairesinol can find larger commercial use.

Considerable amounts of lignans are found in coniferous trees. The type of lignans differs in different species and the amounts of lignans vary in different parts of the trees. The typical lignans in heart wood of spruce (Picea abies) are hydroxymatairesinol (HMR), α-conidendrin, conidendric acid, matairesinol, isolariciresinol, secoisolariciresinol, liovil, picearesinol, lariciresinol and pinoresinol (R. Ekman, “Distribution of lignans in Norway spruce”, Acta Acad. Abo, Ser. B, 39:3, 1-6 (1979). The far most abundant single component of lignans in spruce is hydroxymatairesinol (HMR), about 60 per cent of total lignans, which occurs mainly in unconjugated free form. The lignan concentration in thick roots is 2-3 per cent. Abundance of lignans occurs in the heart wood of branches (5-10 per cent) and twists and especially in the knots, where the amount of lignans may be higher than 10 per cent (R. Ekman, “Analysis of lignans in Norway spruce by combined gas chromatography—mass spectrometry”, Holzforschung 30, 79-85 (1976); R Ekman 1979; S. Willför, J. Hemming, M. Reunanen, C. Eckerman and B. Holmbom, “Hydrophilic and lipophilic extractives in Norway spruce knots”. Presented at 11. ISWPC, Nice, France, 2001). These concentrations are about hundred-fold compared to ground flax powder known as lignan-rich material.

It has been suggested to isolate hydroxymatairesinol from compression-wood fiber. These fibers originate from compression wood of stems and knots (oversized chip fraction) and they are known to weaken the quality of paper (R. Ekman, 1976; S Willför et al., 2001).

It has recently been found that high amounts of hydroxymatairesinol can be produced by extracting finely divided wood material, preferably spruce knotwood, with a polar solvent or solvent mixture and precipitating hydroxymatairesinol from the extract as a complex. Suitable solvents to be used in the extraction step are, for example, pure ethanol or a mixture of ethanol and ethyl acetate. After the extraction step at least part of the solvent is preferably withdrawn before the addition of a complexing agent, which preferable is a carboxylate, such as acetate, of an alkali metal, such as potassium, an earth alkali metal, or ammonium. Such carboxylates form crystallisable adducts with hydroxymatairesinol. An especially preferable complexing agent is potassium acetate, which gives an easily crystallisable potassium acetate adduct of hydroxymatairesinol. This adduct is also rich in the (−) hydroxymatairesinol diastereomer.

A laboratory method for the preparation of matairesinol from hydroxymatairesinol by use of palladium in acetic acid ester has been described by Freudenberg K and Knof L, “Lignanes des Fichtenholzes”. Chem. Ber. 90, 2857-69, 1957.

### OBJECT AND SUMMARY OF THE INVENTION

An object of this invention is a method for the synthesis of matairesinol from a source available in large quantities, namely hydroxymatairesinol, especially hydroxymatairesinol derived from wood.

This invention concerns a method for the preparation of matairesinol from hydroxymatairesinol, either by
(i) catalytic hydrogenolysis of the hydroxy group in 7-position of hydroxymatairesinol, wherein the reaction is carried out in a suitable solvent as a pressurized hydrogenolysis, or
(ii) reduction of hydroxymatairesinol, wherein the reduction is carried out as a hydrogen transfer reaction from a hydrogen donor in the presence of a catalyst.

### BRIEF DESCRIPTION OF THE DRAWINGS

FIGS. 1 and 5 show the concentration evolvement of matairesinol (end product) and the hydroxymatairesinol diastereomers (starting material) as function of time in a pressurized hydrogenolysis using Pd on carbon as catalyst.

FIGS. 2 to 4 show the concentration evolvement of matairesinol (end product) and the hydroxymatairesinol diastereomers (starting material) as function of time in a pressurized hydrogenolysis using Raney Nickel as catalyst.

### DETAILED DESCRIPTION OF THE INVENTION

Hydroxymatairesinol appears as two diastereomers, namely (−) hydroxymatairesinol and (−) allo-hydroxymatairesinol. The word “hydroxymatairesinol”, shall in the definition of this invention be understood to cover any pure geometric isomer or pure stereoisomer or any pure diastereomer or mixture of isomers or diasteromers of the compound. Salts, adducts and complexes of the compound shall also be understood to be covered by the term.

1. Catalytic Hydrogenolysis of the Hydroxy Group in 7-Position of Hydroxymatairesinol in a Suitable Solvent by Pressurized Hydrogenolysis

According to a preferable embodiment, the catalytic hydrogenolysis is a heterogen catalysis.

A suitable catalyst is a metal or metal oxide or a mixture of metals and/or metal oxides. The catalyst can be either a metal or metal oxide powder, or the catalyst can be applied to a carrier. As suitable elements in the carrier can be mentioned Si, Al and C. The carrier can be, for example, solid particles such as powders, granules or extrudates.

The process can be carried out using any known reactor technology. It can thus be a slurry process where the catalyst particles are suspended in the liquid phase. Alternatively, a structured catalysis can be used. In this alternative, no filtration of the catalyst is needed. As examples of structured structured catalyses can be mentioned use of monolith technologies, packed columns, trickle-beds, Sulzer-type catalysts or fiber-bound catalysts.

Preferably, the catalyst is Pd, Pt, Ni, Rh, Ru, Co, a Raney-type catalyst such as Raney-Ni, or a oxide of the aforementioned elements. Mixtures of these metals and/or their oxides can also be used.

As especially suitable catalysts can be mentioned Raney-Ni or palladium on carbon (PdC).

In laboratory scale without any separate hydrogen source, the reaction can be carried out, for example, by use of Raney-Nickel catalyst in excess. In production scale, however, a separate hydrogen source (hydrogen gas) shall be used so as to reduce the amount of the expensive catalyst.

The term “pressurized hydrogenolysis” shall be understood to include any suitable pressure above normal atmosphere pressure, or the range about 2 to 200 bar. A preferable pressure range is, however, 5-70 bar, or even more preferably 15-70 bar.

The temperature is preferably kept in the range from 50 to 150° C.

As suitable solvents can be mentioned, for example, alcohols, ethers, esters, ketones, hydrocarbons or halogenated hydrocarbons. According to a preferred embodiment, the solvent is an alcohol such as ethanol, 2-propanol or a mixture thereof.

2. Reduction of Hydroxymatairesinol by Hydrogen Transfer Reaction from a Hydrogen Donor in the Presence of a Catalyst.

Ammonium formate has been found to be a particularly suitable hydrogen donor. The publication Ram, S. and Spicer L. D.; Tetrahedron Letters 29 (1988) 3741-3744 disclose the use of this agent as hydrogen donor for the reduction of aldehydes and ketones in a catalytic hydrogen transfer reaction. The use of this agent in other hydrogen transfer reactions is disclosed in S. Ram and R. E. Ehrekaufer; Synthesis, 91 (1988). However, none of these publications indicates that this agent would be usefull in the reduction of a benzylic hydroxyl group.

Richard C. Larock, Comprehensive Organic Transformations, A Guide to Functional Group Preparations, Wiley-VCH 1989, presents a summary of methods for the reduction of a benzylic hydroxyl group; i.e. reduction of the group “aryl-CH(OH)—” to “aryl-CH₂—”. Many of the reagents disclosed in this book would not be suitable for use in the reduction of hydroxymatairesinol to matairesinol. The presence of strong acids would most likely result in formation of conidendrine.

Hydrogen donors by use of which the reduction could be carried out under mild conditions would most likely be useful. As examples of such hydrogen donors can be mentioned, in addition to ammonium formate, cyclohexene, a borohydride such as sodium borohydride, or a silane, especially a halotrialkylsilane.

A suitable catalyst is a metal or metal oxide or a mixture of metals and/or metal oxides. The catalyst can be either a metal or metal oxide powder, or the catalyst can be applied to a carrier. As suitable elements in the carrier can be mentioned Si, Al and C. The carrier can be, for example, solid particles such as powders, granules or extrudates.

Preferably, the catalyst is Pd, Pt, Ni, Rh, Ru, Co, a Raney-type catalyst such as Raney-Ni, or an oxide of the aforementioned elements. Mixtures of these metals and/or their oxides can also be used.

As especially suitable catalysts can be mentioned palladium on carbon (PdC).

As suitable solvents can be mentioned, for example, glacial acetic acid, or aqueous ethanol at slightly acidic pH.

Although both of the hydroxymatairesinol diasteromers can be used for the synthesis according to both of the alternatives (i) and (ii) of this invention, it may be desirable to use the (−) hydroxymatairesinol diastereomer. According to one preferable embodiment, the hydroxymatairesinol is in the form of a complex or adduct, precipitated from an extract obtained by extracting wood material with a polar solvent, such as a potassium carboxylate adduct of hydroxymatairesinol.

The hydroxymatairesinol or its adduct in the starting material to be used in the synthesis does not necessarily need to be purified from other components. However, the starting material should not contain components detrimental to the catalyst.

The invention will be illuminated by the following non-restrictive Experimental Section.

### Experimental Section

In the Examples below, the following abbreviations are used: MR=matairesinol; HMR1=the (−) allohydroxymatairesinol diastereomer; HMR2=the (−) hydroxymatairesinol diastereomer; and HMR=a mixture of the (−) allohydroxymatairesinol diastereomer and the (−) hydroxymatairesinol diastereomer.

Examples 1-2 are reference examples describing the catalytic hydrogenolysis at atmospheric pressure. Examples 3-9 describe the process according to this invention, where pressurized conditions are used. Example 10 illustrates the hydrogen transfer.

### EXAMPLE 1

A mixture of 1 g HMR and 5 g Raney Nickel (50% slurry in water, Acros) was stirred in 50 ml ethanol at 50° C. After 24 h, 5 g Raney-Ni was added and the mixture was stirred for 24 h more.

The mixture was then filtered and the solvent removed under reduced pressure. Analyses by GC and GC-MS showed that only 3% of HMR was transformed into matairesinol and no other products could be detected.

### EXAMPLE 2

HMR (300 mg) was dissolved in 5 ml benzene and 5 ml THF. To the solution was added 300 mg Raney-Ni (50% slurry) at room temp. The mixture was stirred for 1 h and then H₂ was allowed to flow trough the mixture. The mixture was then heated to 50° C. and stirred for 24 h under H₂ at atmospheric pressure (balloon). Additional Raney-Ni (300 mg) was added and the stirring was continued under H₂ for 5 days. The mixture was then filtered and the solvent was removed under reduced pressure. Analyses by GC and GC-MS showed that approximately 2% of HMR was converted to MR.

The Raney Nickel (50% slurry in water) used in Examples 1 and 2 was washed several times with ethanol before it was added to the reaction mixture.

### EXAMPLE 3

An isothermal, laboratory scale, stainless steel pressure autoclave (no baffles) having an internal diameter of 64 mm and a length of 103 mm was filled with 150 ml of 1,2-dichlorethane in which 19.35 g of HMR (humid) was dissolved. 1.5 g of 10% Pd on active carbon (Acros Chemicals) catalyst was inserted into the reactor vessel together with the reaction mixture and heating was switched on. The mixture was flushed with hydrogen (99.999% pure, AGA Oyj) for 2 minutes to remove oxygen from the vessel. During the heating period the stirrer was not engaged. After 1-2 hours heating with an oil bath the reactor reached the desired reaction temperature of 50° C. (323 K).

The stirrer was switched on (1000 rpm) and this was considered the initial start of the hydrogenation batch. The pressure was adjusted to 80 PSI (approx. 5.5 bar).

The reaction was allowed to proceed for four (4) hours and small amounts of samples were withdrawn from the reaction mixture every 30 min. for later analysis by means of GC (gas chromatography). The samples (a few milliliters) were obtained through a 5 μm metallic sinter filter by cracking a sample valve, immediately wrapped into an aluminium folio to protect them from light exposure and transferred to a freezer (−20° C., 253 K).

After 4 hours the initial MR concentration of 0.18 wt-% increased to 58.9 wt-%, whereas the initial HMR content (HMR1 36.4 wt-%, HMR2 61.7 wt-%) was reduced to 8.5 and 28.8 wt-% respectively (HMR1, HMR2).

### EXAMPLE 4

An isothermal, laboratory scale, stainless steel pressure autoclave (no baffles) having an internal diameter of 64 mm and a length of 103 mm was filled with 100 ml of HPLC-grade EtOH (ethyl alcohol) in which 1.15 g of HMR (dried in a vacuum oven overnight) was dissolved. 0.1725 g of 10% Pd on active carbon (Acros Chemicals) catalyst was inserted into the reactor vessel together with the reaction mixture and heating was switched on. The mixture was flushed with hydrogen (99.999% pure, AGA Oyj) for 2 minutes to remove oxygen from the vessel. During the heating period the stirrer was not engaged. After 1-2 hours heating with an oil bath the reactor reached the desired reaction temperature of 65° C. (338 K). The stirrer was switched on (1000 rpm) and this was considered the initial start of the hydrogenation batch. The pressure was adjusted to 80 PSI (approx. 5.5 bar).

The reaction was allowed to proceed for 175 minutes and small amounts of samples were withdrawn from the reaction mixture at scheduled intervals for later analysis by means of GC (gas chromatography). The samples (a few milliliters) were obtained through a 5 μm metallic sinter filter by cracking a sample valve, immediately wrapped into an aluminium folio to protect them from light exposure and transferred to a freezer (−20° C., 253 K).

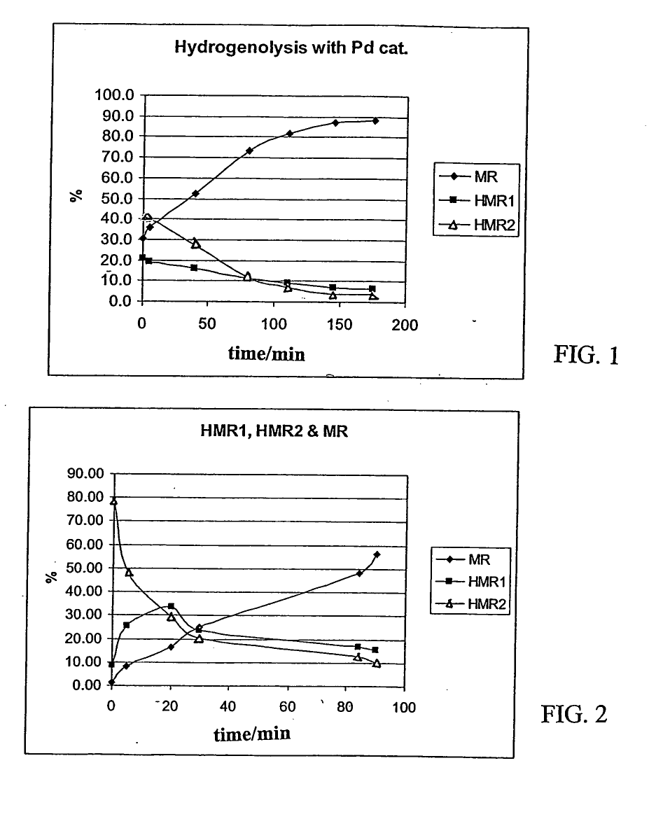

After 175 minutes the initial MR concentration of 30.6 wt-% increased to 88.2 wt-%, whereas the initial HMR content (HMR1 20.6 wt-%, HMR2 40.9 wt-%) was reduced to 6.19 and 3.81 wt-% respectively (HMR1, HMR2). The concentration evolvement as a function of time is displayed in FIG. 1.

### EXAMPLE 5

An isothermal, laboratory scale, stainless steel pressure autoclave (with baffles) having an internal diameter of 64 mm and a length of 103 mm was filled with 100 ml of HPLC-grade EtOH (ethyl alcohol) in which 1 g of the potassium acetate-HMR adduct (1:1) was dissolved. 1.35 g of commercial Raney nickel (promoted with molybdenum) (Activated metals) catalyst was inserted into the reactor vessel together with the reaction mixture and heating was switched on. The mixture was flushed with hydrogen (99.999% pure, AGA Oyj) for 2 minutes to remove oxygen from the vessel. During the heating period the stirrer was not engaged. After 2 minutes heating with an electrical coil the reactor (equipped with a cooling coil and temperature controller) reached the desired reaction temperature of 100° C. (373 K). The stirrer was switched on (1700 rpm) and this was considered the initial start of the hydrogenation batch. The pressure was adjusted to 420 PSI (approx. 29 bar).

The reaction was allowed to proceed for 90 minutes and small amounts of samples were withdrawn from the reaction mixture at scheduled intervals for later analysis by means of GC (gas chromatography). The samples (a few milliliters) were obtained through a 5 μm metallic sinter filter by cracking a sample valve, immediately wrapped into an aluminium folio to protect them from light exposure and transferred to a freezer (−20° C., 253 K).

After 90 minutes the initial MR concentration of 1.4 wt-% increased to 56.4 wt-%, whereas the initial HMR content (HMR1 8.5 wt-%, HMR2 78.5 wt-%) was reduced to 15.6 and 9.8 wt-% (HMR1, HMR2). The concentration evolvement as a function of time is displayed in FIG. 2.

### EXAMPLE 6

An isothermal, laboratory scale, stainless steel pressure autoclave (no baffles) having an internal diameter of 64 mm and a length of 103 mm was filled with 100 g of HPLC-grade EtOH (ethyl alcohol) in which 3 g of the potassium acetate-HNM adduct (1:1) was dissolved. 3 g of commercial Raney nickel (promoted with molybdenum) (Activated metals) catalyst was inserted into the reactor vessel together with the reaction mixture and heating was switched on. The mixture was flushed with hydrogen (99.999% pure, AGA Oyj) for 2 minutes to remove oxygen from the vessel. During the heating period the stirrer was not engaged. After 10 minutes heating with an electrical coil the reactor (equipped with a cooling coil and temperature controller) reached the desired reaction temperature of 100° C. (373 K). The stirrer was switched on (1150 rpm) and this was considered the initial start of the hydrogenation batch. The pressure was adjusted to 280 PSI (approx. 19 bar).

The reaction was allowed to proceed for 240 minutes and small amounts of samples were withdrawn from the reaction mixture at scheduled intervals for later analysis by means of GC (gas chromatography). The samples (a few milliliters) were obtained through a 5 μm metallic sinter filter by cracking a sample valve, immediately wrapped into an aluminium folio to protect them from light exposure and transferred to a freezer (−20° C., 253 K).

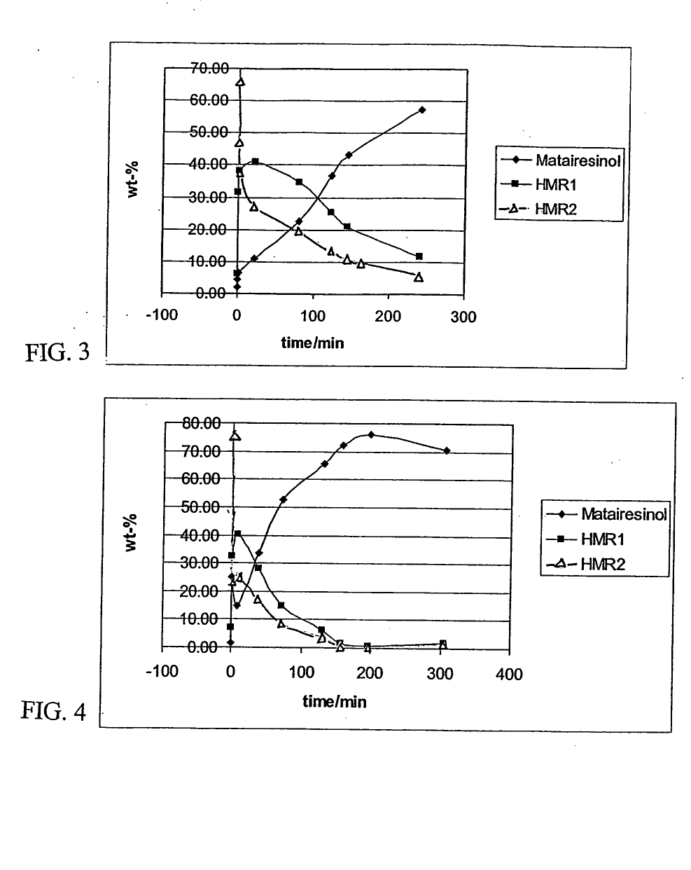

After 240 minutes the initial MR concentration of 1.95 wt-% increased to 57.5 wt-%, whereas the initial HMR content (HMR1 6.1 wt-%, HMR2 66 wt-%) was reduced to 11.6 and 5.9 wt-% (HMR1, HMR2). The concentration evolvement as a function of time is displayed in FIG. 3.

### EXAMPLE 7

An isothermal, laboratory scale, stainless steel pressure autoclave (no baffles) having an internal diameter of 64 mm and a length of 103 mm was filled with 100 g of HPLC-grade EtOH (ethyl alcohol) in which 2.38 g of the potassium acetate-HMR adduct (1:1) was dissolved. 2.38 g of commercial Raney nickel (promoted with molybdenum) (Activated metals) catalyst was inserted into the reactor vessel together with the reaction mixture and heating was switched on. The mixture was flushed with hydrogen (99.999% pure, AGA Oyj) for 2 minutes to remove oxygen from the vessel. During the heating period the stirrer was not engaged. After 10 minutes heating with an electrical coil the reactor (equipped with a cooling coil and temperature controller) reached the desired reaction temperature of 120° C. (393 K). The stirrer was switched on (1150 rpm) and this was considered the initial start of the hydrogenation batch. The pressure was adjusted to 250 PSI (approx. 17 bar).

The reaction was allowed to proceed for more than 300 minutes and small amounts of samples were withdrawn from the reaction mixture at scheduled intervals for later analysis by means of GC (gas chromatography). However, it turned out that the maximum yield of MR was obtained at around 200 min.

The samples (a few milliliters) were obtained through a 5 μm metallic sinter filter by cracking a sample valve, immediately wrapped into an aluminium folio to protect them from light exposure and transferred to a freezer (−20° C., 253 K).

After 197 minutes the initial MR concentration of 1.65 wt-% increased to 76 wt-%, whereas the initial HMR content (HMR1 6.91 wt-%, HMR2 75.6 wt-%) was reduced to 0.60 and 0.53 wt-% (HMR1, HMR2). The concentration evolvement as a function of time is displayed in FIG. 4.

### EXAMPLE 8

An isothermal, laboratory scale, stainless steel pressure autoclave (no baffles) having an internal diameter of 64 mm and a length of 103 mm was filled with 100 g of HPLC-grade EtOH (ethyl alcohol) in which 3.0 g of the potassium acetate-HMR adduct (1:1) was dissolved. 0.3 g of commercial Pd on activated carbon (Acros) catalyst was inserted into the reactor vessel together with the reaction mixture and heating was switched on. The mixture was flushed with hydrogen (99.999% pure, AGA Oyj) a couple of times to remove oxygen from the vessel. During the heating period the stirrer was not engaged. After 10 minutes heating with an electrical coil the reactor (equipped with a cooling coil and temperature controller) reached the desired reaction temperature of 100° C. (373 K). The stirrer was switched on (1150 rpm) and this was considered the initial start of the hydrogenation batch. The pressure was adjusted to 435 PSI (approx. 30 bar).

The reaction was allowed to proceed for more than 240 minutes and small amounts of samples were withdrawn from the reaction mixture at scheduled intervals for later analysis by means of GC (gas chromatography). However, it turned out that the maximum yield of MR was obtained at around 210 min.

The samples (a few milliliters) were obtained through a 5 μm metallic sinter filter by cracking a sample valve, immediately wrapped into an aluminium folio to protect them from light exposure and transferred to a freezer (−20° C., 253 K).

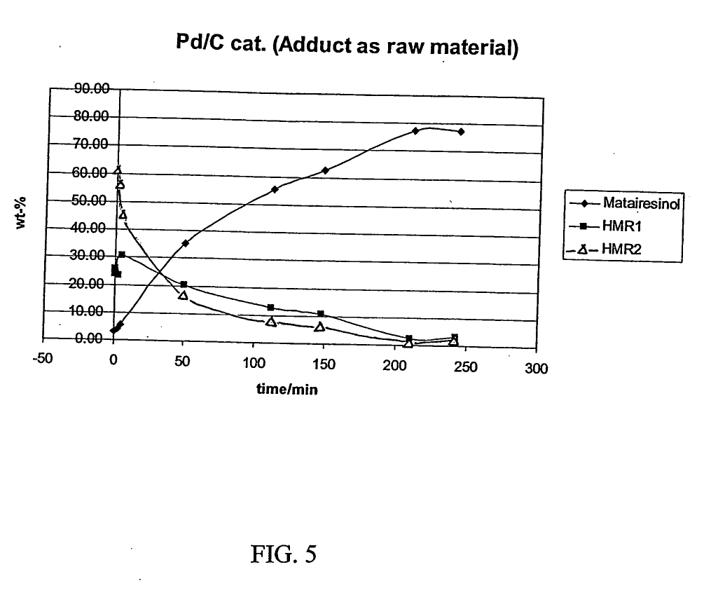

After 210 minutes the initial MR concentration of 3.36 wt-% increased to 77.4 wt-%, whereas the initial HMR content (HMR1 25.7 wt-%, HMR2 61.1 wt-%) was reduced to 3.05 and 2.15 wt-% (HMR1, HMR2). The concentration evolvement as a function of time is displayed in FIG. 5.

### EXAMPLE 9

An isothermal, laboratory scale, stainless steel pressure autoclave (with baffles) having an internal diameter of 64 mm and a length of 103 mm was filled with 150 mL of 1,2-dichloroethane (DCE, J. T. Baker p.a.). HMR 1.5 g and Pd—C 10% (Fluka) 0.3 g catalyst were added to the reaction vessel. The reaction mixture was warmed with an electrical coil up to 50° C. while it was flushed with N₂ (99.999% pure, AGA Oyj) to remove oxygen from the vessel (equipped with a cooling coil and temperature controller). The reaction mixture was stirred first at 500 rpm and after the temperature had reached 36° C., at 1000 rpm. When the reaction mixture reached temperature 50° C., H₂ (99.999% pure, AGA Oyj) was introduced to the reaction mixture followed by N₂ to make sure that all oxygen had been removed from the reaction mixture. After this the hydrogenolysis was performed by introducing again H₂ to the vessel. The pressure was adjusted to 8 bar. The reaction was allowed to proceed for 240 minutes after which the reaction mixture was filtered through a filter paper. According to GC-MS (HP-5890 Series II Gas Chromatograph equipped with a 5971 A Mass Selective Detector; column 30 m×0.25 mm×0.25 μm HP-1MS) the conversion of HMR to MR was quantitative.

### EXAMPLE 10

Reduction of hydroxymatairesinol to matairesinol by hydrogen transfer reaction using ammonium formate as a hydrogen donor.

Hydroxymatairesinol (4.0 g, 11 mmol) is dissolved in glacial acetic acid (80 ml). Ammonium formate (2.0 g, 32 mmol) and 10% palladium on carbon (0.4 g) are added under nitrogen atmosphere. The mixture is refluxed for two hours. The catalyst is removed by filtration through a celite pad and washed with ethanol. The solvent is evaporated. The residue is dissolved in ethyl acetate and washed with dilute sodium carbonate and water. The solvent is evaporated to give 2.85 g (74%) of matairesinol.

¹H NMR (CDCl₃): 2.39-2.66 (m, 4H), 2.81-3.01 (m, 2H), 3.81 and 3.815 (2s, 6H), 3.88 (dd, 1H), 4.15 (dd, 1H), 5.54 and 5.56 (2s, 2H), 6.40-6.61 (m, 4H), 6.80 (d, 1H), 6.82 (d, 2H)

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

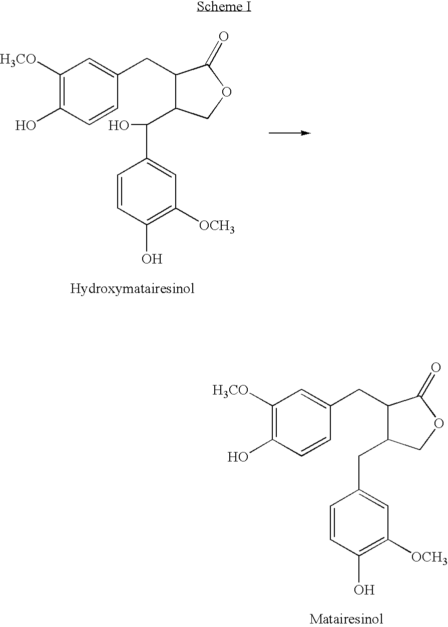

1-16. (canceled)

17. Method for the preparation of matairesinol from hydroxymatairesinol, either by
(i) catalytic hydrogenolysis of the hydroxy group in 7-position of hydroxymatairesinol, wherein the reaction is carried out in a suitable solvent as a pressurized hydrogenolysis, or
(ii) reduction of hydroxymatairesinol, wherein the reduction is carried out as a hydrogen transfer reaction from a hydrogen donor in the presence of a catalyst.

18. The method of claim 17, alternative (i), wherein the catalytic hydrogenolysis is a heterogen catalysis.

19. The method of claim 18, wherein the reaction is carried out at a pressure in the range of 5 to 70 bar.

20. The method of claim 18, wherein the solvent is an alcohol, ether or hydrocarbon.

21. The method of claim 20, wherein the solvent is an alcohol such as ethanol or 2-propanol.

22. The method of claim 17, alternative (ii), wherein the hydrogen donor is ammonium formate, cyclohexene, a borohydride or a silane.

23. The method of claim 22, wherein the reaction is carried out under mild conditions.

24. The method of claim 22, wherein the solvent is glacial acetic acid, or aqueous ethanol at slightly acidic pH.

25. The method of claim 17, wherein the catalyst is a metal or metal oxide or a mixture of metals and/or metal oxides.

26. The method of claim 25, wherein the catalyst is Pd, Pt, Ni, Rh, Ru, Co, a Raney-type catalyst such as Raney-Ni, or an oxide of the aforementioned elements, or a mixture thereof.

27. The method of claim 26, wherein the catalyst is Raney-Nickel.

28. The method of claim 26, wherein the catalyst is palladium on carbon (PdC).

29. The method of claim 17, wherein the hydroxymatairesinol is a mixture of the (−) hydroxymatairesinol diastereomer and the (−) allohydroxymatairesinol diastereomer.

30. The method of claim 17, wherein the hydroxymatairesinol is the (−) hydroxymatairesinol diastereomer.

31. The method of claim 17, wherein the hydroxymatairesinol is in the form of a complex or adduct, precipitated from an extract obtained by extracting wood material with a polar solvent.

32. The method according to claim 31, wherein the adduct is a potassium carboxylate adduct of hydroxymatairesinol.
